(19) United States
(12) Patent Application Publication
(10) Pub. No.: US 2021/0047657 A1
(43) Pub. Date: Feb. 18, 2021

(54) METHODS OF AAV VECTOR PRODUCTION BY MODULATING DEUBIQUITINATING ENZYME ACTIVITY

(71) Applicant: OREGON HEALTH & SCIENCE UNIVERSITY, Portland, OR (US)

(72) Inventors: Hiroyuki Nakai, Portland, OR (US); Swapna Kollu, Portland, OR (US)

(21) Appl. No.: 17/079,266

(22) Filed: Oct. 23, 2020

Related U.S. Application Data
(63) Continuation of application No. PCT/US2019/029466, filed on Apr. 26, 2019.
(60) Provisional application No. 62/672,248, filed on May 16, 2018.
(60) Provisional application No. 62/663,229, filed on Apr. 26, 2018.

(51) Int. Cl. C12N 15/86; C12N 9/48; C12N 7/00

(52) U.S. Cl. CPC C12N 15/86; C12N 9/485; C12N 2750/14152; C12N 7/00; C12N 2750/14143; C12Y 304/19012

(57) ABSTRACT

Disclosed are host cells that have modulated DUB gene product activity and methods of using such host cells for increased AAV vector production yield. The disclosure also provides fusion proteins that comprise a DUB domain from a DUB gene product and a viral protein domain, and methods of increasing production of AAV vectors from a host cell that comprise expressing at least one of these fusion proteins in the host cell.

### RELATED APPLICATIONS

This application is a continuation of International Application No. PCT/US2019/029466, which was filed on Apr. 26, 2019, and also claims the benefit of the earlier filing date of both U.S. Provisional Patent Application No. 62/663,229, which was filed on Apr. 26, 2018, and U.S. Provisional Patent Application No. 62/672,248, which was filed on May 16, 2018, and each of these applications is hereby incorporated by reference in its entirety.

### STATEMENT REGARDING FEDERALLY SPONSORED RESEARCH

This invention was made with government support under Grant No. R01 NS088399 awarded by the National Institutes of Health. The government has certain rights in the invention.

### TECHNICAL FIELD

This disclosure relates generally to methods for AAV vector production for use in gene therapy applications. More specifically, this disclosure relates to methods to enhance the yield of AAV vector production by modulation of a host cell deubiquitination pathway.

### BACKGROUND

Adeno-associated virus (AAV) has shown promise as a vector for human gene therapy. To date, AAV1, 2, 4, 5, 6, 8, 9, rh10, and several capsid-engineered mutants have been successfully used in clinical trials of gene therapy for the treatment of hemophilia A and B, inborn errors of metabolism, ocular diseases, neurodegenerative diseases, muscular dystrophies, and more (1). Among these AAV strains, AAV2 is the prototype serotype and has been used in many of the early clinical studies. More recently, AAV9 has been shown to mediate robust transduction in the central nervous system (CNS) following intravenous injection, leading to success of gene therapy for spinal muscular atrophy, a life-threatening motor neuron disease that affect infants and children (2). Despite such tremendous progress in AAV vector-mediated gene therapy, several important obstacles still remain to be overcome for further advancement of AAV vector-mediated gene therapy and its broader applications to a wide range of more common human diseases. One of the long-standing challenges in AAV vector-mediated gene therapy is an inherent difficulty in cost-effective manufacturing of AAV vectors on a large scale for human use (3). Although AAV vector products that have met regulatory requirements have been manufactured and used in human clinical trials, the recent withdrawal of the first AAV vector commercial product, Glybera, from the market has highlighted the difficulty in sustaining AAV vector products in the clinic for treating a broader spectrum of patients, due in part to an inherent difficulty in cost-effective manufacturing of high-quality AAV vectors on a large scale for human use (3,4).

Several methods have been shown to be effective to some degree in increasing AAV vector yield on a per-cell basis, which include: mild hypothermia treatment of AAV packaging cells (10), hyperosmotic treatment of transfected cells at vector production (11), manipulation of the cap gene pre-mRNA splice donor sites to enhance splicing (12), forced expression of hsa-miR342 (AAVpro® Helper Free System, Takara Bio Inc.), suppression of Y-box binding protein (YB1) (13), attenuation of transgene expression during vector production (14), and the use of a nonconventional combination of the AAV Rep protein and the ITR that are both derived from AAV serotype 3 (AAV3) (15). Although improvement in AAV vector production is in urgent need in order to meet anticipated patient demand, it has remained challenging to further increase the vector yield due to the paucity of knowledge regarding the mechanisms of capsid assembly.

Factors from the AAV, adenovirus and host cells all interact during the process of AAV vector production. These interactions hold the key to increasing capsid production yields; however, how these factors interact in the capsid assembly process is poorly understood. Viral proteins required for capsid formation are the structural proteins VP1, VP2 and VP3 and the assembly activating protein (AAP), the recently discovered non-structural protein that plays a key role in capsid assembly (16). The roles and functions of AAP in the process of capsid assembly have started being unraveled (6-8,16-23). However, what host cell proteins participate in the capsid assembly process and how they interact with AAV viral proteins, VP1, VP2, VP3 and AAP, remain largely unknown.

### SUMMARY

Disclosed are methods for enhancing the yield of AAV production on a per-cell basis by manipulating the deubiquitinating enzyme (DUB) pathway in the host cell such as human embryonic kidney 293 cells. The methods described may be useful to produce AAV vectors at higher titers for research and clinical use. AAV vectors produced by the methods described herein can be used to deliver genes to one or more tissues such as brain, heart, lung, liver, kidney, spleen, intestine, pancreas, testis, muscle and fat as well as cells in culture dishes. The disclosed methods include modulation of wild-type or mutant DUBs by various techniques including but not limited to (1) overexpression of the DUBs or introduction of a gain-of-function mutation, (2) suppression of DUB expression or introduction of a loss-of-function mutation, (3) altering the localization of the DUBs, (4) molecular engineering DUBs and DUB domains fused with viral proteins (e.g., AAP) or cellular proteins, and (5) chemical modulation of DUBs.

### BRIEF DESCRIPTION OF THE FIGURES

FIG. 1. Map of the BioID constructs used in this study. BirA*-AAP2 fusion constructs BirA* was placed either to the N-terminus of AAP2 (the middle 3 constructs) or to the C-terminus of AAP2 (the bottom three constructs). The top three constructs were BirA* only control constructs. NLS, the nuclear localization signal from SV40; NoLS, nucleolar localization signal; NLS-NoLS, the NLS-NoLS from AAP2.

FIG. 2. Expression and biotin ligase activity of the BirA* proteins. The plasmid DNA constructs shown in FIG. 1 were transfected into HEK293 cells. The cells were exposed to biotin contained in the culture medium 24 hours after transfection and cells were harvested 48h after transfection. The total protein was extracted from the transfected cells and subjected to SDS-PAGE separation followed by immunoblotting. Protein expression levels were assessed by probing with a polyclonal rabbit anti-BirA antibody and enzymatic activity was assessed by streptavidin conjugated with horse radish peroxidase (HRP). Protein expression levels and enzymatic activities of the fusion constructs where BirA* was fused to the AAP2 N-terminus, were higher than those of the constructs where BirA* was fused to the AAP2 C-terminus. Interestingly, the capsid assembly-promoting function was preserved in all the constructs (see FIG. 4).

FIG. 3. Sub-cellular localization of the fusion constructs where BirA* is on the N-terminus of AAP2 in the presence of VP3. Both BirA*-AAP2 (B-AAP2) and BirA*NLS-NoLS-AAP2 (B-NLS/NoLS-AAP2) fusion constructs localize to the nucleolus and successfully translocate the AAV2 VP3 protein to the nucleolus, similar to the AAP2 only control construct. AAV2 VP3 by itself cannot localize to the nucleolus without co-expression of AAP2 (7) (data not shown in this figure).

FIG. 4. A dot blot analysis showing the ability for the BirA*-AAP2 fusion proteins to facilitate AAV2 capsid assembly. The AAP's capsid assembly-promoting function was found to be partially to fully preserved in all the 6 fusion constructs we have created.

FIG. 5. An example of Bio-ID pull-down with streptavidin beads assessed by immunoblotting. Samples were probed with a rabbit polyclonal anti-BirA antibody. The same blots were then stripped of the old probe and re-probed with a mouse monoclonal anti-AAV VP1,2,3 antibody (B1 antibody). AAV2 VP3 was pulled down as a biotin-labeled protein, validating the success of BioID labeling and immunoprecipitation. I, input; S, supernatant; B, precipitants pulled-down with the beads; B-E, beads after elution; and E, eluate. The eluates were subsequently submitted to an LC-MS/MS analysis.

FIG. 6. Predicted protein-protein interactions of the 33 proteins identified by the BioID experiment is shown. STRING was used to draw the protein-protein network. A majority of the proteins are those involved in ribosome biogenesis, which form a cluster around the center. Of particular interest is USP36, a nucleolar localizing deubiquitinating enzyme that is functionally remote from the majority of the other nucleolar proteins forming a dense core network. Nodes representing importin 5 and nucleophosmin, which have also been identified as BioID-labeled proteins, are shown.

FIG. 7. Polyubiquitination of AAV2 VP protein and USP36 effects on assembly. (A, B) His-tagged ubiquitin (Ub) was overexpressed in HEK293 cells together with or without the indicated AAV1 or AAV2 protein. His-Ub-tagged proteins were pulled-down with Ni-NTA beads and probed with each indicated antibody by western blot (WB). VP proteins were polyubiquitinated in the absence of AAP. However, polyubiquitinated VP proteins were not present at appreciable levels in the presence of AAP. Anti-pan ubiquitin antibody, sc-8017; D, DMSO; M, MG132. (C) A dot blot showing the effect of overexpression of USP36 and USP36C131A (DN) on capsid assembly. AAV1 or AAV2 VP3 only viral particles were produced in HEK293 cells overexpressing either the wild-type USP36 or the USP36DN or in HEK293 cells transfected with an empty plasmid as a mock control. AAV VP3 only viral particles were produced in all the three conditions; however, the yields of AAV viral particle production were significantly decreased when the USP36 was inhibited by the overexpression of USP36DN. Overexpression of the wild-type USP36 did not have any obvious impact on the AAV particle production yields. (D) Overexpression of USP36DN enhanced polyubiquitination of AAV2 VP3 proteins deduced by higher MW-bands in WB, in the absence of AAP2. AAV VP3 only viral particles were produced in HEK293 cells overexpressing either the wild-type USP36 or the USP36DN or in HEK293 cells transfected with an empty plasmid as a mock control, in the presence or absence of AAP2. and in the presence of overexpressed wild-type USP36 or USP36DN. Anti-VP antibody was used for WB. MG132 was not used for this experiment. Closed arrowheads, VP3; open arrowheads, ubiquitinated VP3. WB signals representing polyubiquitinated (Ubn) VP proteins are indicated with brackets.

FIG. 8. Knock-down of endogenous USP36 expression impairs production of AAV2 VP123 viral particles and VP3 only viral particles (note: VP123 viral particles are those composed of VP1, VP2 and VP3 subunits at an appropriate stoichiometry ratio produced with a standard AAV Rep/Cap helper plasmid). AAV2 VP123 and VP3 only viral particles were produced in HEK293 cells transfected either with a scrambled shRNA-expressing plasmid or a human USP36-specific shRNA-expressing plasmid (USP36 shRNA-3 in reference (27)). VP123 and VP3 only viral particle yields produced in each condition were determined by a quantitative dot blot. In the graph, each yield is normalized of the value obtained from each scrambled shRNA control. Error bars, SEM.

FIG. 9. Overexpression of a nucleolar-excluded USP36 mutant (USP36NoE) enhances the yield of AAV2 vector production. (A) Maps of the USP36WT or USP36NoE-expressing plasmid and the control empty plasmid under the control of the CMV-IE enhancer-promoter. USP36WT is the wild-type USP36 while USP36NoE is devoid of the 16 amino acids (positions from 1078 to 1049) that are rich in basic amino acids. (B) Effects of USP36WT or USP36NoE overexpression on the production of AAV2 VP123 viral particle and AAV2 VP3 only viral particles in HEK293 cells. AAV2 AV123 and VP3 only viral particles were produced in HEK293 cells transfected either of the three plasmids described in Panel A. All the values are normalized with the empty plasmid control values. Error bars, SEM. One-way ANOVA with post-hoc Tukey honesty significant different (HSD) test (n=11 for VP123 to 5 for VP3) is used.

### DETAILED DESCRIPTION

To begin comprehensively elucidating the interactions between host cell proteins and AAV viral components in the process of capsid assembly, we first employed the standard approach that combines co-immunoprecipitation (co-IP) and liquid chromatography tandem-mass spectrometry (LC-MS/MS). To this end, we expressed FLAG-tagged AAP2 (FLAG-AAP2) in the presence or absence of AAV2 VP3 protein, immunoprecipitated (IP'ed) FLAG-AAP2 with anti-FLAG antibody-coated beads, and sought to identify co-immunoprecipitated proteins by LC-MS/MS. We performed this experiment twice, only to find AAV2 VP3 and importin 5 as meaningful proteins co-IP'ed with AAP2. We speculated that the reason why the co-IP approach proved challenging in identifying AAP-interacting proteins was due to the weak or transient nature of protein interactions that might be lost during co-IP. To overcome this potential problem, we next employed BioID, a proximity labeling-based MS approach that provide spatially restricted proteome data (24-26). In the BioID approach, a promiscuous bacterial biotin ligase mutant (BirA*) is fused to a bait protein either at the N- or C-terminus. Such a bait-BirA* fusion protein can biotin-label any proteins residing in close proximity to the bait in cells, which can be pulled down by streptavidin beads under stringent conditions. This approach has successfully identified a set of host cell protein candidates that potentially interact with AAP2. Among them, we have confirmed that USP36 plays a role in AAV2 VP protein assembly into capsid by exerting its deubiquitinating (DUB) enzyme activity. This disclosure describes methods to enhance AAV vector production by manipulating the DUB pathway.

The present disclosure provides host cells that have modulated DUB gene product activity and methods of using such host cells for increased AAV vector production yield. The term “AAV vector” as used herein means any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. The term “AAV vector” may be used to refer to an AAV type viral particle (or virion) comprising a nucleic acid molecule encoding a protein of interest.

Additionally, the AAVs disclosed herein may be derived from various serotypes, including combinations of serotypes (e.g., “pseudotyped” AAV) or from various genomes (e.g., single-stranded or self-complementary). In particular embodiments, the AAV vectors disclosed herein may comprise desired proteins or protein variants. A “variant” as used herein refers to an amino acid sequence that is altered by one or more amino acids. The variant may have “conservative” changes, wherein a substituted amino acid has similar structural or chemical properties, e.g., replacement of leucine with isoleucine. Alternatively, a variant may have “nonconservative” changes, e.g., replacement of a glycine with a tryptophan. Analogous minor variations may also include amino acid deletions or insertions, or both.

In particular embodiments, the AAV vector may be a human serotype AAV vector. In such embodiments, a human AAV may be derived from any known serotype, e.g., from AAV1, AAV2, AAV4, AAV6, or AAV9.

The AAV vectors disclosed herein may include a nucleic acid encoding a protein of interest. In various embodiments, the nucleic acid also may include one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid may comprise a promoter region operably linked to the coding sequence to cause or improve expression of the protein of interest in infected cells. Such a promoter may be ubiquitous, cell- or tissue-specific, strong, weak, regulated, chimeric, etc., for example, to allow efficient and stable production of the protein in the infected tissue. The promoter may be homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. Other promoters that may be used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the phosphoglycerate kinase (PGK) promoter and the β-actin promoter.

In some embodiments of the AAV vectors disclosed herein, one or more feedback elements may be used to dampen over-expression of the protein of interest. For example, some embodiments of the AAV vectors may include one or more siRNA sequences that would target the exogenous transcript. In other embodiments, the AAV vector may include one or more additional promoters that may be recognized by inhibitory transcription factors. In various embodiments, the AAV vectors disclosed herein may comprise a construct that may create a homoeostatic feedback loop that may maintain expression levels of the protein of interest at a physiological level.

In various embodiments, the AAV vectors disclosed herein can comprise a nucleic acid that may include a leader sequence allowing secretion of the encoded protein. In some embodiments, fusion of the transgene of interest with a sequence encoding a secretion signal peptide (usually located at the N-terminal of secreted polypeptides) may allow the production of the therapeutic protein in a form that can be secreted from the transduced cell. Examples of such signal peptides include the albumin, the β-glucuronidase, the alkaline protease or the fibronectin secretory signal peptides.

As described herein, effective and long term expression of therapeutic proteins of interest in brain, heart, lung, skeletal muscle, kidney, spleen, or pancreas can be achieved with non-invasive techniques, through peripheral administration of certain AAV vectors. Such peripheral administration may include any administration route that does not necessitate direct injection into brain, heart, lung, skeletal muscle, kidney, spleen, or pancreas. More particularly, peripheral administration may include systemic injections, such as intramuscular, intravascular (such as intravenous,) intraperitoneal, intra-arterial, or subcutaneous injections. In some embodiments, peripheral administration also may include oral administration (see, e.g., WO96/40954), delivery using implants, (see, e.g., WO01/91803), or administration by instillation through the respiratory system, e.g., using sprays, aerosols or any other appropriate formulations.

In various embodiments, the desired doses of the AAV vectors may be adapted by the skilled artisan, e.g., depending on the disease condition, the subject, the treatment schedule, etc. In some embodiments, from 10⁵ to 10¹² viral genomes are administered per dose, for example, from 10⁶ to 10¹¹, from 10⁷ to 10¹¹, or from 10⁸ to 10¹¹. In other embodiments, exemplary doses for achieving therapeutic effects may include virus titers of at least about 10⁵, 10⁶, 10⁷, 10⁸, 10⁹, 10¹⁰ or 10¹¹ viral genomes or more. Virus titer may also be expressed in terms of transducing units, which may be readily calculated by those of skill in the art.

In various embodiments, the AAV vectors disclosed herein may be administered in any suitable form, for instance, either as a liquid solution or suspension, as a solid form suitable for solution or suspension in liquid prior to injection, as a gel or as an emulsion. The vectors may be formulated with any appropriate and pharmaceutically acceptable excipient, carrier, adjuvant, diluent, etc. For instance, for injection, a suitable carrier or diluent may be an isotonic solution, a buffer, sterile and pyrogen-free water, or, for instance, a sterile and pyrogen-free phosphate-buffered saline solution. For inhalation, the carrier may be in particulate form.

The vectors may be administered in a “therapeutically-effective” amount, e.g., an amount that is sufficient to alleviate (e.g., decrease, reduce) at least one of the symptoms associated with a disease state, or to provide improvement in the condition of the subject. In some embodiments, repeated administrations may be performed, for instance using the same or a different peripheral administration route and/or the same vector or a distinct vector.

In some embodiments, the present disclosure provides a host cell for increased AAV vectors production yield in which the host cell comprises a modulated DUB gene product activity. In certain embodiments, the modulated DUB gene product activity comprises at least one of (a) alteration of expression of the DUB gene or (b) alteration of the subcellular localization of the DUB gene product. The modulated DUB gene product activity may include expression of a DUB gene that is not normally expressed in the host cell. Alternatively, the modulated DUB gene product activity may include overexpression or suppression of expression of a DUB gene in the host cell. In some embodiments, the modulated DUB gene product activity comprises a loss of the DUB gene activity in the host cell.

The DUB gene may be wild-type or mutant. When referring to a gene, the term “wild-type” is used in its ordinary sense and is defined as a gene that has the same protein-coding nucleotide sequence as the corresponding gene in that host cell. For gene sequences that are polymorphic, “wild-type” refers to the sequence of the most common form of the gene. The term “mutant” is also used in its ordinary sense and is defined as a gene that does not have the same protein-coding nucleotide sequence as the corresponding gene in that host cell. A mutation may be one or more of a change in one or more nucleotides, a deletion of one or more nucleotides, or an insertion of one or more nucleotides. The term may also refer to an alteration in the number of copies of a gene or in one or more of the elements that control its expression.

In some embodiments, the modulated DUB gene product activity comprises alteration of the subcellular localization of the DUB gene product. The DUB gene product may comprise a heterologous localization peptide. In some embodiments, the heterologous localization peptide may be derived from a different gene of the same species. In other embodiments, the heterologous localization peptide may be derived from a gene from a different species, including a virus. The localization peptide may cause the DUB protein to localize to the nucleus, nucleolus, cytoplasm, or any other desired subcellular location. Several subcellular localization peptides are known in the art.

The DUB gene product may be fused with a portion of an AAP or other viral protein. Alternatively, a portion or domain of the DUB gene product maybe fused to a portion or the whole of an AAP protein or a VP protein. The DUB gene, or the fused portion or domain thereof, may be wild-type or mutant. The AAP or VP protein, or the fused portion thereof, may be wild-type or mutant.

The DUB gene may be any gene that encodes deubiquitinating enzyme. In some embodiments, the DUB gene encodes a deubiquitinating enzyme from the cysteine protease class of DUB genes. The DUB gene may encode a ubiquitin-specific protease. Alternatively, the DUB gene may encode an ovarian tumor protease. In certain embodiments, the DUB gene encodes the ubiquitin-specific protease USP36. The USP36 may be human, or it may be derived from another organism.

The present disclosure also provides methods of producing AAV vectors that use the host cells described above. In some embodiments, such methods comprise introducing an AAV vector plasmid into the host cell and culturing the host cell to produce AAV vectors.

AAV vectors may be produced by a triple transfection method in HEK293 cells wherein the AAV rep and cap genes, a transgene cassette to be packaged in the AAV particle, and a minimal set of genes from the human adenovirus type 5 (which is a helper virus) are supplied in trans as plasmid DNA to the host HEK293 cells (5). AAV vector production in HEK 293 cells may be performed using the following illustrative description of an adenovirus-free plasmid transfection method (5) with modification on a scale of 1 to 2-liter culture. In brief, HEK 293 cells (AAV-293), purchased from Agilent (AAV-293 cells) are grown in Dulbecco's modified Eagle's medium (DMEM) (Lonza, Basel, Switzerland) supplemented with 10% fetal bovine serum (FBS), L-glutamine, and penicillin-streptomycin. Immediately before plasmid DNA transfection, the culture media is changed to serum-free media. The following three plasmids are transfected at a 1:1:1 ratio in HEK293 cells using the standard polyethyleneimine (PEI) (1 mg/mL) DNA transfection procedure at a DNA:PEI weight ratio of 1:2. The three plasmids used for AAV vector production are: (1) pHELPER (an adenovirus helper plasmid), (2) pHLP-AAVx (an AAV helper plasmid supplying AAV2 Rep and AAVx capsid proteins, where x=AAV serotypes and mutants), and (3) an AAV vector plasmid containing a transgene expression cassette placed between the two AAV2 inverted terminal repeats (ITRs). If AAV vector production requires AAP supplementation, a fourth plasmid, pCMV-AAPx (6-8), may be included at a 1:1:1:1 ratio. In certain embodiments, in which AAV vector is produced with supplementation of either wild-type or genetically modified DUB, a fifth plasmid (for example, a plasmid expressing a DUB or genetically engineered DUB under the control of the CMV promoter) at a 1:1:1:1:1 ratio. Five days after transfection, both media and cells are harvested. The harvested media and cells undergo one cycle of freezing and thawing and the cell debris is removed by centrifugation. The culture medium supernatants are made to 8% polyethylene glycol (PEG) 8000 and 0.5 M NaCl, incubated on ice for 3 h, and spun at 10,000×g for 30 min to precipitate viral particles. The pellets are resuspended in a resuspension buffer such as phosphate-buffered saline (PBS) or a buffer containing 50 mM Tris-HCl (pH 8.5) and 2 mM MgCl2, treated with an endonuclease (such as Benzonase) for 1 h, and subjected to purification by two rounds of cesium chloride (CsCl) density-gradient ultracentrifugation (9) followed by dialysis with PBS with 0.001% Pluronic F68. The final viral preparations are made in PBS/5% sorbitol/0.001% Pluronic F68 and stored −80° C. until use. The AAV vector titers are determined by a quantitative dot blot assay (8). Other methods that may be used for AAV vector production are also available including methods that use baculovirus and insect cells, methods that use ion-exchange chromatography or affinity chromatography for vector purification, and methods that use quantitative real-time PCR for AAV vector titration.

In certain embodiments, the present disclosure provides methods of producing AAV vectors comprising the steps of introducing an adenovirus helper plasmid, an AAV helper plasmid, and an AAV vector plasmid into a cell of a host cell line, wherein the host cell line has a modulated DUB gene product activity; and culturing the host cell to produce AAV vectors. The host cell line may be a stable cell line that comprises one or more genetic alterations that modulate expression of a DUB gene, affect the activity of a DUB gene product, or alter the subcellular localization of a DUB gene product. In some embodiments, the host cell line comprises an engineered host cellular genome, wherein the engineered host cellular genome expresses one or more biological molecules capable of affecting the expression level, activity, and/or subcellular localization of one or more DUB genes. In other embodiments, the host cell line comprises an engineered host cellular genome that comprises modifications of the nucleotide sequence of one or more DUB genes or DUB gene control sequences (promoters, enhancers, etc.).

In some embodiments, the host cell line comprises an extrachromosomally replicating vector genome, wherein the extrachromosomally replicating vector genome expresses one or more biological molecules capable of affecting the activity of the DUB gene. The extrachromosomally replicating vector genome may, for example, include a DUB gene under the control of a heterologous promoter, a DUB gene whose product localizes to a different subcellular location than its normal cellular counterpart, or a DUB gene that is fused to a portion of a viral protein.

In some embodiments, methods of producing AAV vectors may further comprise the step of treating the host cell with one or more chemical modulators of DUB activity.

The present disclosure also provides fusion proteins that comprise a DUB domain from a DUB gene product and a viral protein domain, and methods of increasing production of AAV vectors from a host cell that comprise expressing at least one of these fusion proteins in the host cell. In some embodiments, the viral protein domain is an AAP protein or a protein derived from an AAP protein. The DUB domain sequence may be wild-type or mutant. The viral domain sequence may be wild-type or mutant. In some embodiments, the fusion protein localizes primarily to a subcellular location that differs from a primary subcellular location of the DUB gene. The phrase “localizes primarily to a subcellular location” means that more than 50% of the fusion protein will be in that particular subcellular location. In certain embodiments, more than about 60%, more than about 70%, more than about 80%, more than about 90%, more than about 95%, or about 99% of the fusion protein will be in that particular subcellular location. The phrase “primary subcellular location of the DUB gene” refers to the particular subcellular location in which more than 50% of the corresponding DUB gene product is normally found. In certain embodiments, more than about 60%, more than about 70%, more than about 80%, more than about 90%, more than about 95%, or about 99% of the corresponding DUB gene product will be found in that particular subcellular location. The subcellular location may, for example, be the nucleus, nucleolus, cytoplasm, or other appropriate subcellular location.

Another aspect of the present disclosure is directed to methods for producing AAV vectors comprising the steps of (1) producing a host cell in which the expression and/or subcellular localization of at least one DUB gene is altered, and (2) culturing the host cell under conditions to produce the AAV vectors.

### EXAMPLES

The following examples are for illustration only. In light of this disclosure, those of skill in the art will recognize that variations of these examples and other embodiments of the disclosed subject matter are enabled without undue experimentation.

### Example 1

### Identification of Host Cell Proteins that Interact with AAP

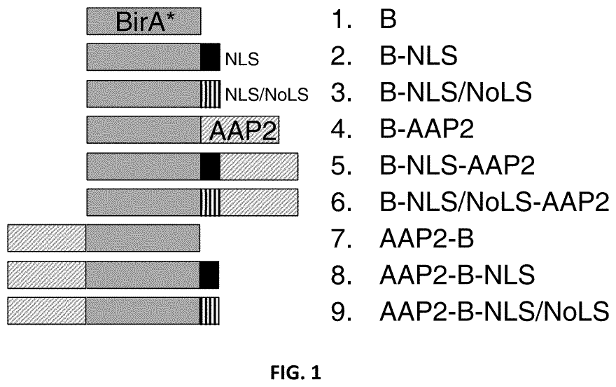

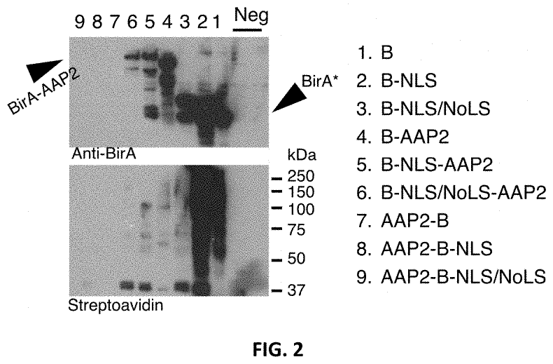

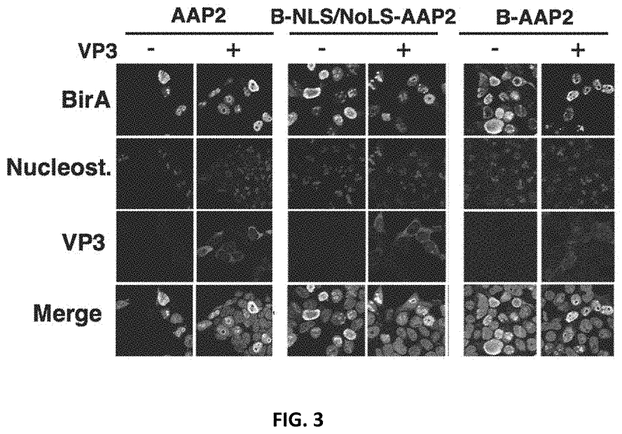

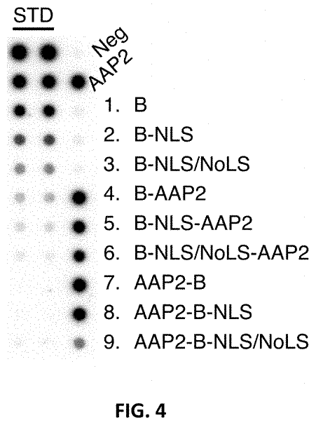

To identify the host cell proteins that interact with AAP, we utilized a proximity-based biotin ligase identification (BioID) method (24-26). In this method, a promiscuous bacterial biotin ligase mutant (BirA*) is fused to the AAP2 protein either at the N- or C-terminus (B-AAP2 or AAP2-B). Such AAP2 and BirA* fusion proteins are expected to biotin-label any proteins residing in close proximity to AAP2 in cells, which can be effectively pulled down by streptavidin beads under stringent conditions such as high salt to reduce non-specific interactions. We constructed a total of 6 AAP2 and BirA* fusion proteins (FIG. 1). Protein expression level and biotin ligase activity of each construct were assessed in HEK293 cells by transient plasmid transfection without AAV2 VP3 co-expression. Protein expression levels of the fusion constructs where BirA* was fused to the AAP2 N-terminus were higher than those of the constructs where BirA* was fused to the AAP2 C-terminus (FIG. 2). Enzymatic activity of the fusion constructs was found to correlate to the protein expression levels (FIG. 2). Subcellular localization of the fusion constructs and their ability to translocate AAV2 VP3 protein were assessed by immunofluorescence microscopy (IF) of HEK293 cells transfected with the non-fusion and fusion BirA* constructs with or without AAV2 VP3 co-expression. The IF study showed that the subcellular localization of AAP2 was not disturbed with any of the fusion construct and the ability for the fusion constructs to translocate AAV2 VP3 protein to the nucleolus was preserved (FIG. 3). Finally, capsid assembly-promoting function of the BirA*-AAP2 fusion proteins where BirA* was fused to the AAP2 N-terminus, were assessed by the VP3-AAP trans-complementation assay we have established (6,7,18). In this assay, HEK293 cells are transfected with the following 5 plasmids, a plasmid expressing AAV VP3 protein, a plasmid expressing AAP protein, a plasmid containing an AAV vector genome with two AAV2 inverted terminal repeats, a plasmid expressing the AAV2 rep gene, and a plasmid expressing a minimal set of the adenovirus type 5 gene products required for AAV vector production (pHelper from Agilent). The ability for AAP to promote capsid assembly is then assessed by quantifying AAV vector genomes packaged in the assembled viral capsid by dot blotting. The result showed that the capsid assembly-promoting function was fully preserved in the BirA*-AAP2 fusion constructs except for one construct, AAP2-B-NLS/NoLS, showing a decreased function (FIG. 4)

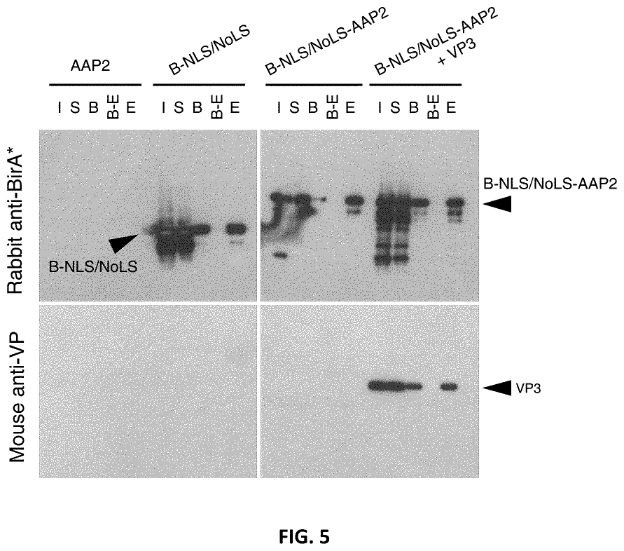

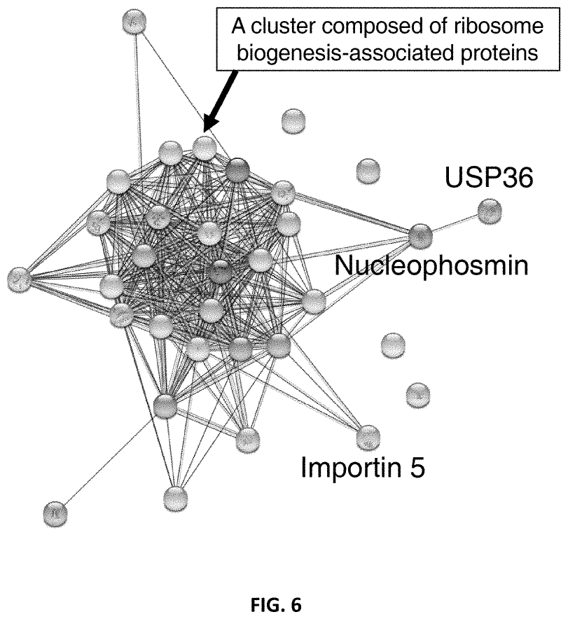

To identify AAP2-interacting host cell proteins, we performed the BioID labeling reaction using the BirA*-AAP2 fusion proteins. We expressed in HEK293 cells the following 4 BirA* constructs (B, B-NLS/NoLS, B-AAP2, B-NLS/NoLS-AAP2) with or without AAV2 VP3 co-expression. Here, B is BirA*, B-NLS/NoLS is BirA* fused with the AAP2 NLS/NoLS at its C-terminus, B-AAP2 is the BirA*-AAP2 fusion construct where BirA* is fused to the N-terminus of AAP2, and B-NLS/NoLS-AAP2 is the BirA*-AAP2 fusion construct where B-NLS/NoLS is fused to the N-terminus of AAP2. Total protein recovered from the transfected HEK293 cells was subjected to a pull-down with streptavidin beads. AAV2 VP3 was pulled down together with the streptavidin beads as a biotin-labeled protein, validating the success of BioID labeling and immunoprecipitation (FIG. 5). The biotin-labeled proteins were eluted from the beads, and the eluates were then subjected to a liquid chromatography-tandem mass spectrometry (LC-MS/MS) analysis. This BioID approach identified at least 33 cellular proteins as candidates for AAP2-interacting proteins in two independent experiments. Gene ontology enrichment analysis of these proteins revealed significant enrichment for nucleolar proteins involved in ribosome biogenesis, validating the proximity-based assay. Predicted protein-protein interactions of the 33 proteins identified by the BioID experiment is shown in FIG. 6. STIRING (28) was used to draw the protein-protein network. Although many of the 33 proteins we have identified may play a role in AAV capsid assembly, we were particularly interested in USP36, a nucleolar-localizing deubiquitinating enzyme (DUB) for the first validation study because USP36 was among the proteins that are remote from the main cluster of ribosome biogenesis-associated proteins and because DUBs are enzymes involved in the ubiquitin-proteasome pathway, the metabolic pathway that is known to play a role in the AAV life cycle and AAV vector transduction (29-33)

### Example 2

### Analysis of Ubiquitination of AAV2 VP Proteins

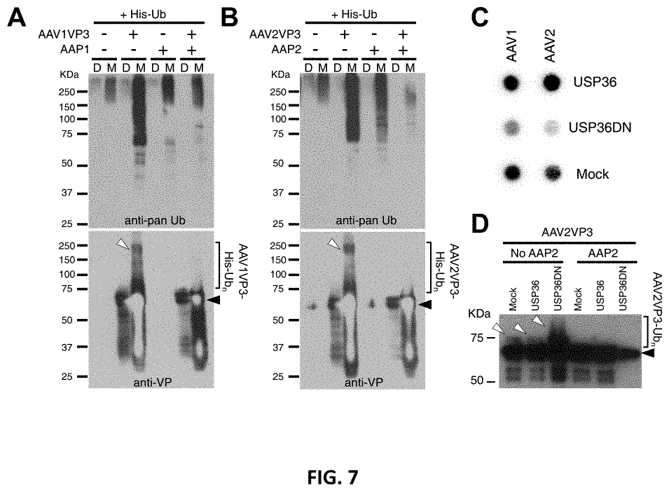

To investigate whether AAV VP proteins become ubiquitinated in the presence or absence of co-expression of AAP, we expressed His-tagged ubiquitin (Ub) (34) in HEK293 cells by transient plasmid transfection together with VP3, AAP or VP3+AAP in the presence or absence of MG132, a proteasome inhibitor. We tested these conditions for both AAV1 VP3 (FIG. 7A) and AAV2 VP3 (FIG. 7B) capsid proteins. His-Ub-tagged proteins were then pulled-down by Ni-NTA beads. His-Ub-tagged proteins were detected by western blot (WB) with anti-pan Ub antibody. This experiment showed substantial accumulation of ubiquitinated proteins in the presence of MG132, demonstrating efficient His-Ub tagging to the proteins that undergo ubiquitination (FIGS. 7A and B, upper panels). When the blots were probed with anti-VP antibody, smeared bands heavier than the mass of VP3 could be detected in both AAV1 and AAV2 samples only in the absence of co-expression of AAP (FIGS. 7A and B, lower panels). Thus, we have concluded that both AAV1 VP proteins and AAV2 VP proteins become polyubiquitinated in the absence of AAP, while in the presence of AAP, ubiquitination of VP proteins was effectively suppressed. Although the mechanism for the AAP-mediated suppression of VP protein ubiquitination has yet to be determined, the following two interpretations are plausible: (1) AAP has a direct role in preventing VP proteins from being ubiquitinated or promoting deubiquitination of polyubiquitinated VP proteins; and (2) AAP promotes VP protein assembly into capsids very rapidly, minimizing the VP protein ubiquitination reaction and converting from VP monomers vulnerable to ubiquitination to VP capsids resistant to ubiquitination (AAP's indirect role).

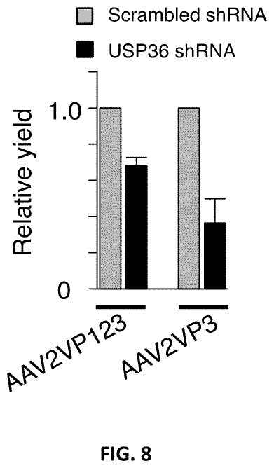

To validate the role of USP36 in the process of AAV1 and AAV2 capsid assembly, we overexpressed the wild-type USP36 and a dominant-negative form of USP36, USP36 C131A (35) in HEK293 cells by transient plasmid DNA transfection when AAV1 or AAV2 VP3 only viral particles were produced. This dominant-negative form has been reported to suppress substantially the DUB enzyme activity of USP36 when overexpressed (35). When the USP36 function was inhibited by the dominant-negative USP36 mutant, both AAV1 and AAV2 viral particle production yields were significantly decreased (FIG. 7C). In addition, polyubiquitination of AAV2 VP3 proteins deduced by higher MW-bands in WB was significantly enhanced (FIG. 12D) in the absence of AAP2. These data demonstrate that USP36 plays a crucial role in AAV1 and AAV2 viral particle production, presumably through deubiquitination of polyubiquitinated AAV capsid proteins that are destined to proteasomal degradation. It has been shown that (1) AAP2 transports AAV2 VP protein monomers to the nucleolus (6,7,16), (2) AAV2 capsid assembly takes place in the nucleolus (36), and (3) USP36 is a nucleolar localizing DUB (37). Taken altogether, we have proposed the following completing hypothetical model that explains how AAP2, AAV2 VP and DUB proteins interact in the process of AAV2 capsid assembly. In this model, AAV2 capsid VP monomer proteins become polyubiquitinated by a not-yet-identified E3 ubiquitin ligase(s) either or both in the cytoplasm and nucleoplasm following their synthesis in cells, and unless the VP proteins become deubiquitinated by a DUB(s), they are destined to disintegration through the ubiquitin-proteasome pathway (UPP), resulting in impaired capsid assembly. In the presence of AAP2, AAP2 stabilizes AAV2 VP monomers by direct binding to VP proteins, and transports VP proteins to the nucleolus to facilitate interactions between polyubiquitinated VP proteins and a DUB(s) in the nucleolus (i.e., USP36). In the nucleolus, polyubiquitin chains on VP proteins are removed by DUB, allowing VP proteins to escape from proteasomal degradation and to assemble into capsids effectively. We have already generated strong evidence that AAP can stabilize VP monomers using assembly-defective AAV capsid VP proteins and functionally competent AAPs derived from cognate or heterologous serotypes in conjunction with a BioID-based approach. As for the process of AAV1 capsid assembly, we assume that the same model can apply. To further confirm the role of USP36 in AAV capsid assembly, we expressed shRNAs against endogenous USP36 to knock-down endogenously-expressed USP36. We used USP36 shRNA-3, a rigorously validated shRNA against USP36 (27). When USP36 shRNA-3 was co-expressed at the production of AAV2 VP123 viral capsids or AAV2 VP3 only capsids in HEK293 cells in the same manner as described above, both VP123 and VP3 viral particle yields were reduced (FIG. 8), supporting our DUB and AAV capsid assembly model.

### Example 3

### Analysis of USP36's Role in Other AAV Serotypes

To investigate the USP36 role in other AAV serotypes, we performed the above described experiments with other AAV serotypes (AAV3B to AAV12, except for AAV10). The result showed that that expression of the dominant-negative USP36 (USP36 C131A) inhibited AAV viral particle production for AAV1, 2, 6 and 8, but not for AAV3B, 4,5,7,9,11 and 12). (Table 1). It is intriguing that all the AAV serotypes that showed the impairment of capsid assembly with USP36 C131A were those whose AAPs are nucleolar associated (7) and the capsid assembly of AAV5 and 9 whose AAPs are nucleolar excluded (7) were not affected. Traditionally, the nucleolus was believed to be the site of capsid assembly (36); however, our study has shown that not all the AAPs accumulate in the nucleolus; not all AAV serotypes use the nucleolus as the site for capsid assembly; and AAPs do not necessarily colocalize with assembled capsids (7). Thus, where, which and how DUBs play a role in AAV capsid assembly likely vary depending on the AAV serotypes. Nonetheless, these data indicate that AAP plays an important role in facilitating the interaction between AAV capsid VP proteins and host cell DUBs.

TABLE 1
Effects of inhibition of the USP36 deubiquitinating
enzyme activity on AAV viral particle production

| | Effect on AAV viral particle production | | | | | | | | | | | |
|---|---|---|---|---|---|---|---|---|---|---|---|---|
| Over- | (−: no effect, +++: significant effect) | | | | | | | | | | | |
| expressed | AA | AA | AA | AA | AA | AA | AA | AA | AA | AA | AA | AA |
| protein | V1 | V2 | V3B | V4 | V5 | V6 | V7 | V8 | V9 | V10 | V11 | V12 |
| WT USP36 | − | − | − | − | − | − | − | − | − | nd | − | − |
| DN USP36 | +++ | +++ | − | − | − | +++ | − | +++ | − | nd | − | − |

WT, wild-type;
DN, dominant-negative;
nd, not determined.
Please note that − and +++ are binary and do not represent the degree of the actual effects.

### Example 4

### Enhancement of AAV2 Vector Production by Overexpressing USP36NoE

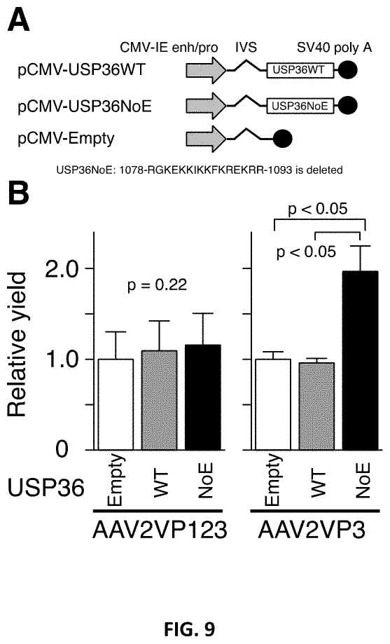

Based on the experimental results described above, the following novel approaches may be used to enhance AAV vector production on a per-cell basis: (1) expression or over-expression of wild-type DUBs (nucleolar, nuclear and cytoplasmic DUBs); (2) expression or overexpression of mutant DUBs (nucleolar, nuclear and cytoplasmic DUBs); (3) relocation of DUBs inside the cell;, (4) expression or over-expression of AAP-DUB fusion proteins, including those that contain only a portion of a DUB protein; and (5) chemical modulation of DUBs by small molecules. Here we provide an example demonstrating how the above described methods could be exploited successfully to improve AAV2 vector particle production. These data suggest that overexpression of nucleolar-excluded USP36 mutant (USP36NoE) enhances AAV2 vector production. The support for this hypothesis is four-fold: (1) AAV2 assembles in the nucleolus and yields much lower titers than many other serotypes; (2) AAV2R585E, AAV5 and AAV9 assemble in the nucleoplasm (outside the nucleolus) and consistently yield 5-10 fold higher titers than AAV2; (3) AAV5 and AAV9 mutants that carry the AAV2-derived RGNR heparin binding motif at the corresponding topological location could not assemble (presumably because their capsid proteins are forcibly brought to a subcellular location where AAV5 or AAV9 do not assemble); however, a single R-to-E mutation in the EGNR fully restored capsid assembly (presumably by restoring subcellular localization in the nucleoplasm); and (4) USP36 is a nucleolar localizing DUB that plays a role in efficient assembly of AAV2 capsids. Without being bound by any particular theory, these data have led us to an inference that a substantial amount of AAV2 VP proteins become ubiquitinated and disintegrated before they reach the nucleolus where deubiquitination of VP proteins takes place for capsid assembly, and this is why nucleolar-assembled serotypes have lower yields than nucleolar-excluded serotypes that can utilize DUBs in the nucleoplasm. Thus, bringing an excess amount of functional USP36 to the nucleoplasm should prevent AAV2VP degradation in the nucleoplasm via its DUB activity and allow more VP proteins to reach the nucleolus, resulting in an increase in AAV2 vector yield. To test this hypothesis, we constructed an USP36NoE with DUB activity based on the information available in literature (35), and overexpressed it when we produced AAV2VP123 and VP3 virions. As we predicted, VP3 virion yields were increased by 2-fold, although overexpression of the wild-type USP36 had no effect (FIG. 9). However, this effect was modest on VP123 virions. To further enhance the USP36NoE′s effect on VP123, a compelling approach is to overexpress a fusion of USP36NoE and AAP2mt26 (6), a nucleolar-excluded, nuclear localized AAP2 mutant. This USP36NoE-AAP2mt26 fusion will facilitates AAV2 VP-DUB interactions in the nucleoplasm, allowing more VP proteins escape from proteasome-mediated degradation and to enter the nucleolus, and therefore further boost the yield of VP123 particularly when the wild-type AAP2 is co-expressed. Alternatively, USP36 could be brought to the cytoplasm either by using its mutants devoid of nuclear localization signal (NLS) or by fusing such mutants with one of the following AAP2 mutants that accumulate in the cytoplasm (i.e., AAP2mt12, mt16, mt17, mt20, mt21, mt22, mt24, mt25, mt27 and mt30 (6)). Alternatively, a portion of USP36 (e.g., only a catalytic domain) can be fused to the wild-type or mutant AAP and co-expressed at AAV vector production. Another approach would be to fuse the full-length or a portion of USP36 with VP protein, followed by an in situ cleavage of USP36-derived protein by expressing a site-specific protease at the site of capsid assembly. These manipulations can be attained either by transient plasmid transfection or transient viral vector infection, by making stable cell lines that harbor the above described manipulations that are incorporated in the host cellular genome, or by making cell lines that persistently carry stably replicating extrachromosomal vector genomes. Alternatively, a chemical agent that modulate USP36 (e.g., a small molecule that enhances USP36 DUB activity) can be supplemented at AAV vector production. Similar approaches may be adopted to any DUBs.

As shown in FIG. 9, overexpression of USP36NoE at the time of AAV2 vector production by transient transfection with pCMV-USP36NoE can boost AAV2 viral particles composed of VP3 capsid proteins and AAV vector genomes by 2 fold (p<0.05, one-way ANOVA followed by post-hoc Tukey HSD test). Although this effect on AAV2 VP123 capsid was modest in the experimental setting we used, the effect will likely be able to be more enhanced by further manipulation of the USP36 DUB pathway. An example is over-expression of AAP2-USP36NoE where AAP2 is fused either to the N-terminus of USP36NpE or to the C-terminus of USP36NoE. In addition, this method could be combined with other methods that have been shown to be effective in increasing AAV vector yield, which may yield additive or synergistic effects. Methods that can be combined with this invention include, but are not limited to, mild hypothermia or hyperosmotic treatment of cells (10,11), manipulation of splicing (12), hsa-miR342 expression, suppression of YB1 (13), attenuation of transgene expression at vector production (14), and use of AAV3 Rep and ITR (15).

In conclusion, we have shown for the first time that the host cell deubiquitination machinery plays an important role in the process of AAV capsid assembly. In addition, our data supports a model in which AAP plays an important role in facilitating the interaction between AAV capsid VP proteins and host cell DUBs. Moreover, we have shown how to exploit this new knowledge to improve AAV vector yields on a per-cell basis. Furthermore, we have demonstrated a successful exemplary application of this knowledge to boost the AAV vector titers in the context of AAV2 VP3 particle production.

### REFERENCES

All references cited in this disclosure are incorporated by reference in their entirety.
1. Wang D, Tai P W L, Gao G. 2019. Adeno-associated virus vector as a platform for gene therapy delivery. Nat Rev Drug Discov doi:10.1038/s41573-019-0012-9.
2. Mendell J R, Al-Zaidy S, Shell R, Arnold W D, Rodino-Klapac L R, Prior T W, Lowes L, Alfano L, Berry K, Church K, Kissel J T, Nagendran S, L'Italien J, Sproule D M, Wells C, Cardenas J A, Heitzer M D, Kaspar A, Corcoran S, Braun L, Likhite S, Miranda C, Meyer K, Foust K D, Burghes A H M, Kaspar B K. 2017. Single-Dose Gene-Replacement Therapy for Spinal Muscular Atrophy. N Engl J Med 377:1713-1722.
3. van der Loo J C, Wright J F. 2016. Progress and challenges in viral vector manufacturing. Hum Mol Genet 25:R42-52.
4. Schnodt M, Buning H. 2017. Improving the Quality of Adeno-Associated Viral Vector Preparations: The Challenge of Product-Related Impurities. Hum Gene Ther Methods 28:101-108.
5. Matsushita T, Elliger S, Elliger C, Podsakoff G, Villarreal L, Kurtzman G J, Iwaki Y, Colosi P. 1998. Adeno-associated virus vectors can be efficiently produced without helper virus. Gene Ther 5:938-945.
6. Earley L F, Kawano Y, Adachi K, Sun X X, Dai M S, Nakai H. 2015. Identification and characterization of nuclear and nucleolar localization signals in the adeno-associated virus serotype 2 assembly-activating protein. J Virol 89:3038-3048.
7. Earley L F, Powers J M, Adachi K, Baumgart J T, Meyer N L, Xie Q, Chapman M S, Nakai H. 2017. Adeno-associated Virus (AAV) Assembly-Activating Protein Is Not an Essential Requirement for Capsid Assembly of AAV Serotypes 4, 5, and 11. J Virol 91.
8. Powers J M, Chang X L, Song Z, Nakai H. 2018. A Quantitative Dot Blot Assay for AAV Titration and Its Use for Functional Assessment of the Adeno-associated Virus Assembly-activating Proteins. J Vis Exp doi:10.3791/56766.
9. Grimm D, Zhou S, Nakai H, Thomas C E, Storm T A, Fuess S, Matsushita T, Allen J, Surosky R, Lochrie M, Meuse L, McClelland A, Colosi P, Kay M A. 2003. Preclinical in vivo evaluation of pseudotyped adeno-associated virus vectors for liver gene therapy. Blood 102:2412-2419.
10. Emmerling V V, Fischer S, Stiefel F, Holzmann K, Handrick R, Hesse F, Horer M, Kochanek S, Otte K. 2016. Temperature-sensitive miR-483 is a conserved regulator of recombinant protein and viral vector production in mammalian cells. Biotechnol Bioeng 113:830-841.
11. Rego M, Hanley L M, Ersing I, Guerin K, Tasissa M, Haery L, Mueller I, Sanders E, M. F. 2018. Improved yield of AAV2 and rAAV2-retro serotypes following sugar supplementation during the viral production phase. bioRxic doi: https://doi.org/10.1101/488585.
12. Farris K D, Pintel D J. 2008. Improved splicing of adeno-associated viral (AAV) capsid protein-supplying pre-mRNAs leads to increased recombinant AAV vector production. Hum Gene Ther 19:1421-1427.
13. Satkunanathan S, Wheeler J, Thorpe R, Zhao Y. 2014. Establishment of a novel cell line for the enhanced production of recombinant adeno-associated virus vectors for gene therapy. Hum Gene Ther 25:929-941.
14. Strobel B, Klauser B, Hartig J S, Lamla T, Gantner F, Kreuz S. 2015. Riboswitch-mediated Attenuation of Transgene Cytotoxicity Increases Adeno-associated Virus Vector Yields in HEK-293 Cells. Mol Ther 23:1582-1591.
15. Ling C, Yin Z, Li J, Zhang D, Aslanidi G, Srivastava A. 2016. Strategies to generate high-titer, high-potency recombinant AAV3 serotype vectors. Mol Ther Methods Clin Dev 3:16029.
16. Sonntag F, Schmidt K, Kleinschmidt J A. 2010. A viral assembly factor promotes AAV2 capsid formation in the nucleolus. Proc Natl Acad Sci USA 107:10220-10225.
17. Grosse S, Penaud-Budloo M, Herrmann A K, Borner K, Fakhiri J, Laketa V, Kramer C, Wiedtke E, Gunkel M, Menard L, Ayuso E, Grimm D. 2017. Relevance of Assembly-Activating Protein for Adeno-associated Virus Vector Production and Capsid Protein Stability in Mammalian and Insect Cells. J Virol 91.
18. Kawano Y, Neeley S, Adachi K, Nakai H. 2013. An experimental and computational evolution-based method to study a mode of co-evolution of overlapping open reading frames in the AAV2 viral genome. PLoS One 8:e66211.
19. Naumer M, Sonntag F, Schmidt K, Nieto K, Panke C, Davey N E, Popa-Wagner R, Kleinschmidt J A. 2012. Properties of the adeno-associated virus assembly-activating protein. J Virol 86:13038-13048.
20. Sonntag F, Kother K, Schmidt K, Weghofer M, Raupp C, Nieto K, Kuck A, Gerlach B, Bottcher B, Muller O J, Lux K, Horer M, Kleinschmidt J A. 2011. The assembly-activating protein promotes capsid assembly of different adeno-associated virus serotypes. J Virol 85:12686-12697.
21. Tse L V, Moller-Tank S, Meganck R M, Asokan A. 2018. Mapping and Engineering Functional Domains of the Assembly Activating Protein of Adeno-Associated Viruses. J Virol doi:10.1128/JVI.00393-18.
22. Maurer A C, Cepeda Diaz A K, Vandenberghe L H. 2019. Residues on Adeno-associated Virus Capsid Lumen Dictate Interactions and Compatibility with the Assembly-Activating Protein. J Virol 93.
23. Maurer A C, Pacouret S, Cepeda Diaz A K, Blake J, Andres-Mateos E, Vandenberghe L H. 2018. The Assembly-Activating Protein Promotes Stability and Interactions between AAVs Viral Proteins to Nucleate Capsid Assembly. Cell Rep 23:1817-1830.
24. Chen C L, Perrimon N. 2017. Proximity-dependent labeling methods for proteomic profiling in living cells. Wiley Interdiscip Rev Dev Biol 6.
25. Kim D I, Birendra K C, Zhu W, Motamedchaboki K, Doye V, Roux K J. 2014. Probing nuclear pore complex architecture with proximity-dependent biotinylation. Proc Natl Acad Sci USA 111:E2453-2461.
26. Kim D I, Jensen S C, Noble K A, Kc B, Roux K H, Motamedchaboki K, Roux K J. 2016. An improved smaller biotin ligase for BioID proximity labeling. Mol Biol Cell 27:1188-1196.
27. Sun X X, He X, Yin L, Komada M, Sears R C, Dai M S. 2015. The nucleolar ubiquitin-specific protease USP36 deubiquitinates and stabilizes c-Myc. Proc Natl Acad Sci USA 112:3734-3739.
28. Szklarczyk D, Morris J H, Cook H, Kuhn M, Wyder S, Simonovic M, Santos A, Doncheva N T, Roth A, Bork P, Jensen L J, von Mering C. 2017. The STRING database in 2017: quality-controlled protein-protein association networks, made broadly accessible. Nucleic Acids Res 45:D362-D368.
29. Braun-Falco M, Eisenried A, Buning H, Ring J. 2005. Recombinant adeno-associated virus type 2-mediated gene transfer into human keratinocytes is influenced by both the ubiquitin/proteasome pathway and epidermal growth factor receptor tyrosine kinase. Arch Dermatol Res 296:528-535.
30. Geoffroy M C, Chadeuf G, Orr A, Salvetti A, Everett R D. 2006. Impact of the interaction between herpes simplex virus type 1 regulatory protein ICP0 and ubiquitin-specific protease USP7 on activation of adeno-associated virus type 2 rep gene expression. J Virol 80:3650-3654.
31. Ros C, Kempf C. 2004. The ubiquitin-proteasome machinery is essential for nuclear translocation of incoming minute virus of mice. Virology 324:350-360.
32. Yan Z, Zak R, Luxton G W, Ritchie T C, Bantel-Schaal U, Engelhardt J F. 2002. Ubiquitination of both adeno-associated virus type 2 and 5 capsid proteins affects the transduction efficiency of recombinant vectors. J Virol 76:2043-2053.
33. Zhong L, Zhao W, Wu J, Li B, Zolotukhin S, Govindasamy L, Agbandje-McKenna M, Srivastava A. 2007. A dual role of EGFR protein tyrosine kinase signaling in ubiquitination of AAV2 capsids and viral second-strand DNA synthesis. Mol Ther 15:1323-1330.
34. Li Y, Sun X X, Elferich J, Shinde U, David L L, Dai M S. 2014. Monoubiquitination is critical for ovarian tumor domain-containing ubiquitin aldehyde binding protein 1 (Otub1) to suppress UbcH5 enzyme and stabilize p53 protein. J Biol Chem 289:5097-5108.
35. Endo A, Kitamura N, Komada M. 2009. Nucleophosmin/B23 regulates ubiquitin dynamics in nucleoli by recruiting deubiquitylating enzyme USP36. J Biol Chem 284:27918-27923.
36. Wistuba A, Kern A, Weger S, Grimm D, Kleinschmidt J A. 1997. Subcellular compartmentalization of adeno-associated virus type 2 assembly. J Virol 71:1341-1352.
37. Endo A, Matsumoto M, Inada T, Yamamoto A, Nakayama K I, Kitamura N, Komada M. 2009. Nucleolar structure and function are regulated by the deubiquitylating enzyme USP36. J Cell Sci 122:678-686.
38. Kategaya L, Di Lello P, Rouge L, Pastor R, Clark K R, Drummond J, Kleinheinz T, Lin E, Upton J P, Prakash S, Heideker J, McCleland M, Ritorto M S, Alessi D R, Trost M, Bainbridge T W, Kwok M C M, Ma T P, Stiffler Z, Brasher B, Tang Y, Jaishankar P, Hearn B R, Renslo A R, Arkin M R, Cohen F, Yu K, Peale F, Gnad F, Chang M T, Klijn C, Blackwood E, Martin S E, Forrest W F, Ernst J A, Ndubaku C, Wang X, Beresini M H, Tsui V, Schwerdtfeger C, Blake R A, Murray J, Maurer T, Wertz I E. 2017. USP7 small-molecule inhibitors interfere with ubiquitin binding. Nature 550:534-538.
39. Lei H, Shan H, Wu Y. 2017. Targeting deubiquitinating enzymes in cancer stem cells. Cancer Cell Int 17:101.
40. Nicholson B, Marblestone J G, Butt T R, Mattern M R. 2007. Deubiquitinating enzymes as novel anticancer targets. Future Oncol 3:191-199.

1. A host cell for producing AAV vectors comprising a modulated DUB gene product activity.

2. The host cell of claim 1, wherein the modulated DUB gene product activity comprises at least one of (a) alteration of expression of the DUB gene or (b) alteration of the subcellular localization of the DUB gene product.

3. The host cell of claim 1, wherein the modulated DUB gene product activity comprises expression of a DUB gene that is not normally expressed in the host cell.

4. The host cell of claim 1, wherein the modulated DUB gene product activity comprises overexpression of a DUB gene in the host cell.

5. The host cell of claim 1, wherein the modulated DUB gene product activity comprises suppression of expression of a DUB gene in the host cell.

6. The host cell of claim 1, wherein the modulated DUB gene product activity comprises a loss of the DUB gene activity in the host cell.

7. The host cell of claim 1, wherein the DUB gene is wild-type.

8. The host cell of claim 1, wherein the DUB gene is mutated.

9. The host cell of claim 1, wherein the modulated DUB gene product activity comprises alteration of the subcellular localization of the DUB gene product.

10. The host cell of claim 9, wherein the DUB gene product comprises a heterologous localization peptide.

11. The host cell of claim 9, wherein the DUB gene is wild-type and the DUB gene product is fused with a wild-type AAP protein.

12. The host cell of claim 9, wherein the DUB gene is mutant and the DUB gene product is fused with a wild-type AAP protein.

13. The host cell of claim 9, wherein the DUB gene is wild-type and the DUB gene product is fused with a mutant AAP protein.

14. The host cell of claim 9, wherein the DUB gene is mutant and the DUB gene product is fused with a mutant AAP protein.

15. The host cell of claim 1, wherein the DUB gene comprises a heterologous subcellular localization targeting peptide.

16. The host cell of any of claims 1-15, wherein the DUB gene encodes a cysteine protease.

17. The host cell of any of claims 1-15, wherein the DUB gene encodes a ubiquitin-specific protease.

18. The host cell of any of claims 1-15, wherein the DUB gene encodes an ovarian tumor protease.

19. The host cell of any of claims 1-15, wherein the DUB gene encodes a human ubiquitin-specific protease.

20. The host cell of claim 19, wherein the ubiquitin-specific protease is human USP36.

21. A method of producing AAV vectors comprising:
Introducing an AAV vector plasmid into the host cell of any of claims 1-15; and
Culturing the host cell to produce AAV vectors.

22. A method of producing AAV vectors comprising:
Introducing an AAV vector plasmid into the host cell claim 16; and
Culturing the host cell to produce AAV vectors.

23. A method of producing AAV vectors comprising:
Introducing an AAV vector plasmid into the host cell of claim 17; and
Culturing the host cell to produce AAV vectors.

24. A method of producing AAV vectors comprising:
Introducing an AAV vector plasmid into the host cell claim 18; and
Culturing the host cell to produce AAV vectors.

25. A method of producing AAV vectors comprising:
Introducing an AAV vector plasmid into the host cell claim 19; and
Culturing the host cell to produce AAV vectors.

26. A method of producing AAV vectors comprising:
Introducing an AAV vector plasmid into the host cell claim 20; and
Culturing the host cell to produce AAV vectors.

27. A method of producing AAV vectors comprising the steps of:
Introducing an adenovirus helper plasmid, an AAV helper plasmid, and an AAV vector plasmid into a cell of a host cell line, wherein the host cell line has a modulated DUB gene product activity; and
Culturing the host cell to produce AAV vectors.

28. The method of claim 27, wherein the host cell line is a stable cell line.

29. The method of claim 28, wherein the host cell line comprises a genetic alteration that modulates expression of the DUB gene.

30. The method of claim 28, wherein the host cell line comprises a genetic alteration in the DUB gene.

31. The method of claim 28, wherein the host cell line comprises a genetic alteration that affects subcellular localization of the DUB gene.

32. The method of claim 28, wherein the host cell line comprises an engineered host cellular genome, wherein the engineered host cellular genome expresses one or more biological molecules capable of affecting the activity of the DUB gene.

33. The method of claim 27, wherein the host cell line comprises an extrachromosomally replicating vector genome, wherein the extrachromosomally replicating vector genome expresses one or more biological molecules capable of affecting the activity of the DUB gene.

34. The method of claim 27, further comprising the step of treating the host cell with one or more chemical modulators of DUB activity.

35. A method of increasing production of AAV vectors from a host cell comprising:
Expressing a fusion protein in the host cell, wherein the fusion protein comprises a DUB domain from a DUB gene product and a viral protein domain.

36. The method of claim 35, wherein the viral protein is an AAP protein.

37. The method of claim 35, wherein the DUB domain is wild-type and the viral protein domain is wild-type.

38. The method of claim 35, wherein the DUB domain is wild-type and the viral protein domain is mutant.

39. The method of claim 35, wherein the DUB domain is mutant and the viral protein domain is wild-type.

40. The method of claim 35, wherein the DUB domain is mutant and the viral protein domain is mutant.

41. The method of claim 35, wherein the fusion protein localizes primarily to a subcellular location that differs from a primary subcellular location of the DUB gene.

42. A method for producing AAV vectors comprising:
Producing a host cell in which at least one of the expression or subcellular localization of at least one DUB gene is altered; and
Culturing the host cell under conditions to produce the AAV vectors.

43. The method of claim 42, wherein the DUB gene comprises a heterologous subcellular localization targeting peptide.

44. The method of claim 42, wherein the DUB gene encodes a cysteine protease.

45. The method of claim 42, wherein the DUB gene encodes a ubiquitin-specific protease.

46. The method of claim 42, wherein the DUB gene encodes an ovarian tumor protease.

47. The method of claim 42, wherein the DUB gene encodes a ubiquitin-specific protease.

48. The method of claim 47, wherein the ubiquitin-specific protease is USP36.
